(19) United States
(12) Patent Application Publication
(10) Pub. No.: US 2024/0216704 A1
(43) Pub. Date: Jul. 4, 2024

(54) HEADER BORE LABELING

(71) Applicant: Cardiac Pacemakers, Inc., St. Paul, MN (US)

(72) Inventors: James Michael English, Cahir (IE); Moira B. Sweeney, St. Paul, MN (US); Robert Allen Jones, Lake Elmo, MN (US); Benjamin J. Haasl, Forest Lake, MN (US); Daniela Guerrera, Clonmel (IE)

(21) Appl. No.: 18/540,388

(22) Filed: Dec. 14, 2023

Related U.S. Application Data
(60) Provisional application No. 63/436,014, filed on Dec. 29, 2022.

(51) Int. Cl. A61N 1/375

(52) U.S. Cl. CPC A61N 1/3754; A61N 1/37512; A61N 1/365

(57) ABSTRACT

Systems and methods are disclosed to affix or print an identifier material on a lead port assembly to identify a type of the lead port assembly before placement in a header of an implantable medical device.

### CLAIM OF PRIORITY

This application claims the benefit of U.S. Provisional Application No. 63/436,014, filed on Dec. 29, 2022, which is hereby incorporated by reference in its entirety.

### TECHNICAL FIELD

This document relates generally to medical devices and more particularly to systems and methods to identify a lead port in a lead port assembly of an implantable medical device.

### BACKGROUND

Medical devices can be implanted or implantable in a body of a patient, such as to monitor patients, including detecting or sensing physiologic information from the patient, such as one or more of heart sounds, respiration (e.g., respiratory rate (RR), tidal volume (TV), etc.), impedance (e.g., thoracic impedance, cardiac impedance, cutaneous impedance, etc.), pressure (e.g., blood pressure), cardiac activity (e.g., heart rate, cardiac electrical information, etc.), chemical (e.g., electrolyte), physical activity, posture, plethysmography, or one or more other physiologic information of the patient, and, in certain examples, provide therapy to the patient in clinical and ambulatory settings. Implantable medical devices (IMDs) can include cardiac rhythm management (CRM) devices, such as pacemakers, cardiac resynchronization devices, cardioverters, defibrillators, drug delivery devices, or one or more other medical devices implanted or implantable within a body of, or subcutaneously to, a patient.

Implantable medical devices often include a hermetically sealed housing containing electronic circuitry of the implantable medical device (e.g., one or more signal processing or control circuits, telemetry circuits, therapy circuits, power management circuits, etc.) and a power source, and one or more lead ports in a header outside of the hermetically sealed housing to couple one or more leads having one or more electrodes or other sensors positioned at various locations in or near a heart of the patient, such as in one or more of the atria or ventricles, to the implantable medical device. Separate from, or in addition to, the one or more electrodes or other sensors of the leads, the implantable medical device can include one or more electrodes or other sensors (e.g., a pressure sensor, an accelerometer, a gyroscope, a microphone, etc.) powered by a power source in the implantable medical device. The one or more electrodes or other sensors of the leads, the implantable medical device, or a combination thereof, can be configured detect physiologic information from the patient, or provide one or more therapies or stimulation to the patient.

Lead ports include various numbers and configurations of electrical contacts for communicating electrical signals into and out of the implantable medical device, such as between the electronic circuitry of the implantable medical device and one or more electrodes coupled to the one or more leads, for example, depending on the type of medical device, the function being performed by the medical device, and the specific lead or leads to be coupled thereto. In certain examples, individual lead ports can include multiple individual components stacked and connected in various configurations to form a number of different stacked bores having different desired physical and electrical configurations. The present inventors have recognized, among other things, a need to improve marking of individual lead ports to reduce cost and complexity of manufacture and assembly of medical device lead ports, medical device headers, and medical devices in general.

### SUMMARY

Systems and methods are disclosed to affix or print an identifier material on a strain relief segment at a proximal end of a bore of a lead port assembly to identify a type of the lead port assembly before placement in a header of an implantable medical device.

An example (e.g., “Example 1”) of subject matter (e.g., a system, a medical device system, etc.) may comprise a lead port assembly for placement in a header of an implantable medical device, the lead port assembly comprising proximal and distal ends, the proximal end comprising a bore configured to receive a proximal end of a lead and an electrical contact configured to couple to an electrical contact of the lead when the lead is inserted into the lead port assembly and an identifier material, separate from the lead port assembly, affixed to or printed on the lead port assembly, comprising a marking to identify a type of the lead port assembly.

In Example 2, the subject matter of Example 1 may optionally be configured such that the lead port assembly comprises a strain relief segment at the proximal end of the bore having a first length, wherein the electrical contact is located distal to the strain relief segment on the lead port assembly and the identifier material is separate from the strain relief segment and the lead port assembly, and affixed to or printed on the strain relief segment of the lead port assembly to identify the type of the lead port assembly.

In Example 3, the subject matter of any one or more of Examples 1-2 may optionally be configured such that the identifier material includes a carrier material comprising the marking, wherein the carrier material is separate from the strain relief segment and the lead port assembly and is affixed to the strain relief segment to identify the type of the lead port assembly.

In Example 4, the subject matter of any one or more of Examples 1-3 may optionally be configured such that the carrier material is affixed to an outer surface of the strain relief segment with an adhesive.

In Example 5, the subject matter of any one or more of Examples 1˜4 may optionally be configured such that the identifier material is affixed to or printed on a side portion of the strain relief segment viewable, through material of the header, from a side of the header.

In Example 6, the subject matter of any one or more of Examples 1-5 may optionally be configured such that the identifier material is affixed to or printed on a top portion of the strain relief segment and the top portion of the strain relief segment is opposite a top of a housing of the implantable medical device, between a top of the header and the top of the housing.

In Example 7, the subject matter of any one or more of Examples 1-6 may optionally be configured such that the strain relief segment comprises a tube shape at the proximal end of the bore having an opening commensurate to a diameter of the lead at the strain relief segment when the lead is fully inserted into the lead port assembly to relieve strain on electrical connections of or associated with the lead port assembly, wherein the first length is along the lead port assembly from the proximal end of the bore.

In Example 8, the subject matter of any one or more of Examples 1-7 may optionally be configured such that the lead port assembly comprises a distal end to contact the proximal end of the lead when the lead is fully inserted into the lead port assembly, wherein the distal end comprises a mechanical retention feature to engage and retain the proximal end of the lead when the lead is fully inserted into the lead port assembly.

In Example 9, the subject matter of any one or more of Examples 1-8 may optionally be configured to include the header and a housing of the implantable medical device, wherein the lead port assembly is a generic lead port assembly for placement in the header, the identifier material is affixed to or printed on the lead port assembly to identify the type of the lead port assembly after the lead port assembly is coupled to a housing of the implantable medical device before incorporation in the header, and the type of the lead port assembly depends at least in part on electrical connections between the lead port assembly and the implantable medical device.

An example (e.g., “Example 10”) of subject matter (e.g., a method) may comprise affixing an identifier material to or printing the identifier material on a lead port assembly for placement in a header of an implantable medical device, wherein the lead port assembly comprises proximal and distal ends, the proximal end comprising a bore configured to receive a proximal end of a lead and an electrical contact configured to couple to an electrical contact of the lead when the lead is inserted into the lead port assembly, and the identifier material comprises a marking to identify a type of the lead port assembly.

In Example 11, the subject matter of any one or more of Examples 1-10 may optionally be configured such that the lead port assembly comprises a strain relief segment at the proximal end of the bore having a first length, the electrical contact is located distal to the strain relief segment on the lead port assembly, the identifier material is separate from the strain relief segment and the lead port assembly, affixing the identifier material to or printing the identifier material on the lead port assembly comprises affixing the identifier material to or printing the identifier material on the strain relief segment of the lead port assembly to identify the type of the lead port assembly.

In Example 12, the subject matter of any one or more of Examples 1-11 may optionally be configured such that affixing the identifier material to or printing the identifier material on the strain relief segment of the lead port assembly comprises affixing the identifier material to or printing the identifier material on the strain relief segment of the lead port assembly prior to forming the header of the implantable medical device.

In Example 13, the subject matter of any one or more of Examples 1-12 may optionally be configured such that the identifier material includes a carrier material comprising the marking, the carrier material is separate from the strain relief segment and the lead port assembly, and affixing the identifier material to or printing the identifier material on the strain relief segment of the lead port assembly comprises printing the marking on the carrier material and affixing the carrier material to the strain relief segment of the lead port assembly to identify the type of the lead port assembly.

In Example 14, the subject matter of any one or more of Examples 1-13 may optionally be configured such that affixing the carrier material to the strain relief segment comprises affixing the carrier material to an outer surface of the strain relief segment using an adhesive.

In Example 15, the subject matter of any one or more of Examples 1-14 may optionally be configured such that affixing the identifier material to or printing the identifier material on the strain relief segment of the lead port assembly comprises affixing the identifier material to or printing the identifier material on a side portion of the strain relief segment viewable through material of the header from a side of a header.

In Example 16, the subject matter of any one or more of Examples 1-15 may optionally be configured such that affixing the identifier material to or printing the identifier material on the strain relief segment of the lead port assembly comprises affixing the identifier material to or printing the identifier material on a top portion of the strain relief segment, and the top portion of the strain relief segment is opposite a top of a housing of the implantable medical device, between a top of the header and the top of the housing.

In Example 17, the subject matter of any one or more of Examples 1-16 may optionally be configured such that the strain relief segment comprises a tube shape at the proximal end of the bore having an opening commensurate to a diameter of the lead at the strain relief segment when the lead is fully inserted into the lead port assembly to relieve strain on electrical connections of or associated with the lead port assembly and the first length is along the lead port assembly from the proximal end of the bore.

In Example 18, the subject matter of any one or more of Examples 1-17 may optionally be configured such that the lead port assembly comprises a distal end to contact the proximal end of the lead when the lead is fully inserted into the lead port assembly and the distal end comprises a mechanical retention feature to engage and retain the proximal end of the lead when the lead is fully inserted into the lead port assembly.

In Example 19, the subject matter of any one or more of Examples 1-18 may optionally be configured such that the lead port assembly is a generic lead port assembly for placement in the header and affixing the identifier material to or printing the identifier material on the lead port assembly comprises affixing the identifier material to or printing the identifier material on the lead port assembly after the lead port assembly is coupled to a housing of the implantable medical device before incorporation in the header.

An example (e.g., “Example 20”) of subject matter (e.g., a method) may comprise identifying a type of a lead port assembly for placement in a header of an implantable medical device using a marking on an identifier material affixed to or printed on the lead port assembly, wherein the lead port assembly comprises proximal and distal ends, the proximal end comprising a bore configured to receive a proximal end of a lead and an electrical contact configured to couple to an electrical contact of the lead when the lead is inserted into the lead port assembly.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1 and 2 illustrate example prior art first and second implantable medical devices having traditional lead ports marked with color and text labeled biocompatible metals adjacent respective lead ports.

FIGS. 3A-3B illustrate an example third implantable medical device including a first lead port assembly having a first identifier to identify a type of the lead port assembly.

FIG. 4 illustrates example lead configurations.

FIG. 5 illustrates an example medical device system.

FIG. 6 illustrates an example patient management system and portions of an environment in which the patient management system may operate.

FIG. 7 illustrates an example method of identifying a type of lead port assembly in a header of an implantable medical device.

FIG. 8 illustrates a block diagram of an example machine upon which any one or more of the techniques (e.g., methodologies) discussed herein may perform.

### DETAILED DESCRIPTION

Traditional lead ports include respective, separate lead ports designed and configured for different leads and respective combinations of leads, electrical contacts, and connections for different implantable medical devices. Implantable medical devices having different lead ports or combinations of lead ports require different headers attached to a housing (or CAN) of the implantable medical device. Traditional lead ports are marked to identify the type of lead configured to be coupled to the lead port during implant.

Traditional lead ports are marked with color and text labeling biocompatible metals, such as by anodizing a surface of a biocompatible metal without altering the biocompatibility of the metal, such as disclosed in the commonly assigned Kane et al. U.S. Pat. No. 9,572,994 titled “Labeled Implantable Medical Devices” (herein, “the ‘994’ patent”), hereby incorporated by reference in its entirety, including its disclosure of positioning a first metal band within a receiving cavity including a first identifier adjacent a lead port within a header, or anodizing one or more identifiers for insertion into the header adjacent the lead port corresponding to the identifier during manufacture of the implantable medical device. In certain examples, the biocompatible metals are inserted into a core defining a bore or a lead port or into a void in the core before the header is overmolded over the core.

Other methods of marking devices and lead bodies include printing biocompatible ink, such as on a lead body or on a device itself in contact with the patient. However, there are several disadvantages associated with such marking, including the time in the manufacture process when such marking will occur, or the place of such marking. In general, ink jet printing is a splattery process. Printing ink inside the header, such as before the header is formed, can require additional processing steps to mask components not intended for ink, additional quality procedures to ensure such printing did not result in ink or debris prior to forming the header, stringent requirements as to the ink used for printing (e.g., biocompatible, non-toxic, non-conductive, handled and cared with certain requirements, etc.), etc. More importantly, such procedures may require batch processing instead of inline processing (e.g., assembly or production line processing), increasing the overall cost and time of manufacture.

In addition, modern lead ports, including multi-element stacked lead port assemblies, are designed in a way to ensure that different combinations of a set of common components can be used to create various permutations of respective combinations of leads and implantable medical devices, such as having leads configured for different locations, a different number of electrode contacts, or having different combinations once incorporated into a medical device (e.g., an implantable medical device having one lead port, two lead ports, three lead ports, four lead ports, etc.). Accordingly, instead of manufacturing lead ports designed for one specific device (such as by manufacturing a specific core for a respective combination of lead ports or a specific implantable medical device), it is advantageous to manufacture and stock the set of generic lead ports or lead port components and to not mark them until it is known what kind of device or lead each respective lead port or component is intended to become.

For example, instead of manufacturing, labeling, and stocking separate right atrium (RA) and right ventricle (RV) lead ports, or creating different cores having specific sets of lead ports and marking the respective lead boars and cores, it is advantageous to manufacture and stock a generic lead port that can be any number of lead ports (e.g., an RA lead port, an RV lead port, etc.) in various combinations or configurations, and then only mark the respective lead port as it is placed in assembly for manufacture into a specific device intended for use. Such procedures can reduce the number of manufacturing cycles, the amount of stock, and the number of components manufactured during each manufacturing cycle, and can reduce waste associated with one component being used at a rate higher or lower than planned for or intended.

Accordingly, the present inventors have recognized, among other things, that lead ports can be labeled to indicate a lead port type during manufacture and assembly of the header or during connection of electrical connections of the lead port to one or more electrical connections of a feedthrough of the housing of the implantable medical device, and not during manufacture of the lead ports themselves, such that the lead ports can remain generic as long as possible before assembly of the implantable medical device.

In an example, a pre-printed label (e.g., a pre-printed sticker or other marker, etc.) can be created in a separate process from manufacture of the lead port or other components of the implantable medical device and attached to or placed on the lead port inline during manufacture and assembly, attachment, or placement of the lead port to the housing (e.g., to the feedthrough) or otherwise during inline assembly (e.g., assembly or production line assembly) of the implantable medical device. Such pre-printed label ensures that such that splatter of a printing process is not an issue during creation of the label, and placement of a pre-printed label can be made inline, such as using a common pick and place process during inline assembly, and does not require transition of assembly from an inline process to a batch process. The pre-printed labels can be printed and placed on a reel, to be picked and placed using a pick and place machine during inline assembly of the header components (e.g., the lead port, the wires between the lead port and the feedthrough, etc.). In this way, the lead port can remain generic until placed in the header for assembly. Additionally, as the header can be epoxied and overmolded (e.g., with clear, transparent, or translucent materials) over the pre-printed label, control of biocompatible label materials and inks for the label is not an issue, as the process of forming the header over the lead core ensures biocompatibility. Legibility, viewable through the material of the header, is the greatest concern. Moreover, because the pre-printed labels are created separately from the pick and place procedure, the size of the labels can be maximized, larger than that which can be printed while in-place during assembly of the header (e.g., printing around a stress relief segment of the lead port having a rounded outer surface requires increased complexity and reduced available printing area as the segment curves, or introducing printing variation as the segment curves away from the printing component). In addition, the pick and place machine can conform to the profile of the portion of the lead port being marked. A pliable material (e.g., rubber) can be shaped to the same curve or profile as the portion of the lead port being marked, applying a constant force over the entire area receiving the pre-printed label, and in certain examples, rolled or pressed to ensure proper position and contact before epoxy or overmolding. Accordingly, the pre-printed label results in a larger, more legible, more easily placed identifier, improving readability and reducing complexity during assembly.

In other examples, the lead port can be laser marked inline during assembly, also avoiding splatter and batch processing, but requiring more sophisticated equipment to laser mark around the portion of the lead

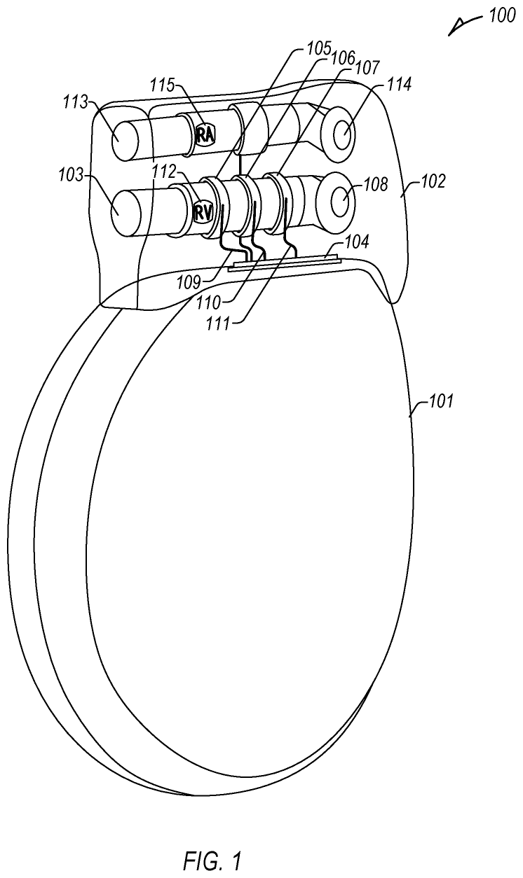

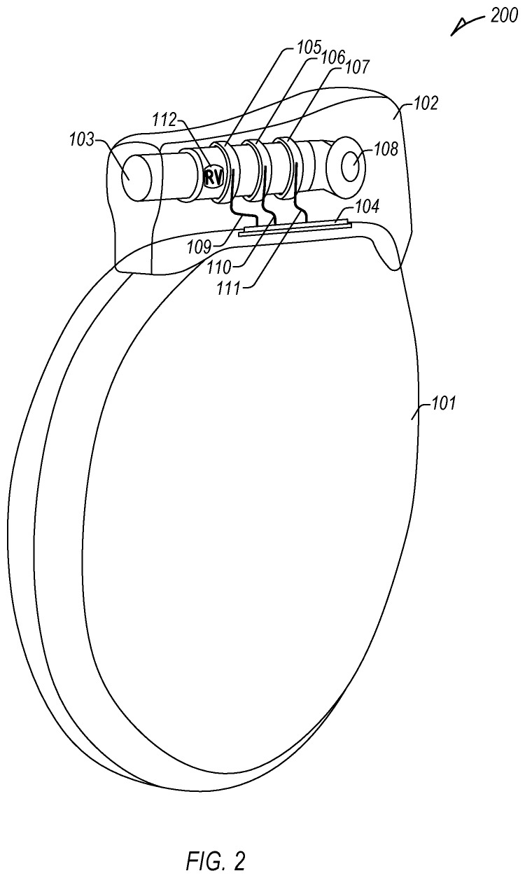

FIGS. 1 and 2 illustrate example prior art first and second implantable medical devices 100, 200 having traditional lead ports marked with color and text labeled biocompatible metals adjacent respective lead ports. Each of the first and second implantable medical devices 100, 200 include a housing 101 (e.g., commonly referred to as a CAN) and a header 102 coupled to the housing 101, including a first lead port 103 coupled to one or more electronic circuits or electronic circuitry in the housing 101 through a feedthrough 104 on top of the housing 101. In other examples, the feedthrough 104 can be located on a side of the housing 101.

The first lead port 103 includes respective electrical connections (e.g., first, second, and third electrical connections 105, 106, 107, etc.) configured to couple to respective electrical connections of a respective lead (not illustrated) once inserted into the first lead port 103 and retained by a first retention element 108 (e.g., a mechanical retention feature, such as a set screw or one or more other anchor, retention component, or locking member, etc.). The first, second, and third electrical connections 105, 106, 107 can be coupled to the feedthrough 104 using respective first, second, and third wires 109, 110, 111. In an example, the feedthrough 104 can include one or more posts or connection points configured to engage or couple to the first, second, and third wires 109, 110, 111 (e.g., pre-formed wires, etc.). In other examples, one or more of the first, second, and third wires 109, 110, 111 can include wires of the feedthrough 104, such as passing through the feedthrough 104 or otherwise coupled to one or more connections of the feedthrough 104, etc.

The first lead port 103 includes a first identifier 112, in this example, an “RV” label having a specific color (e.g., red) identifying the first lead port 103 as a right ventricle (RV) lead port to receive a lead configured to be implanted in or otherwise be associated with a right ventricle of a patient.

The first implantable medical device 100 in FIG. 1 additionally includes a second lead port 113 having corresponding electrical connections, such as those illustrated and described above with respect to the first lead port 103, a second retention element 114, and a second identifier 115, in this example, an “RA” label having a specific color (e.g., gray) identifying the second lead port 113 as a right atrium (RA) lead port to receive a lead configured to be implanted in or otherwise be associated with a right atrium of the patient.

The first and second identifiers 112, 115 include a flat biocompatible metal positioned in the header material (e.g., in a cavity in the header material) between the respective first or second lead port 103, 113 and the outer surface of the header 102, such as illustrated in the ‘994’ patent described above and incorporated by reference herein in its entirety herein.

In contrast to the first implantable medical device 100 described in FIG. 1, the second implantable medical device 200 in FIG. 2 includes only the first lead port 103, and not the second lead port 113 illustrated in FIG. 1. In other examples, implantable medical devices can have one or more other lead ports or number of lead ports (e.g., three lead ports, four lead ports, etc.) in various configurations.

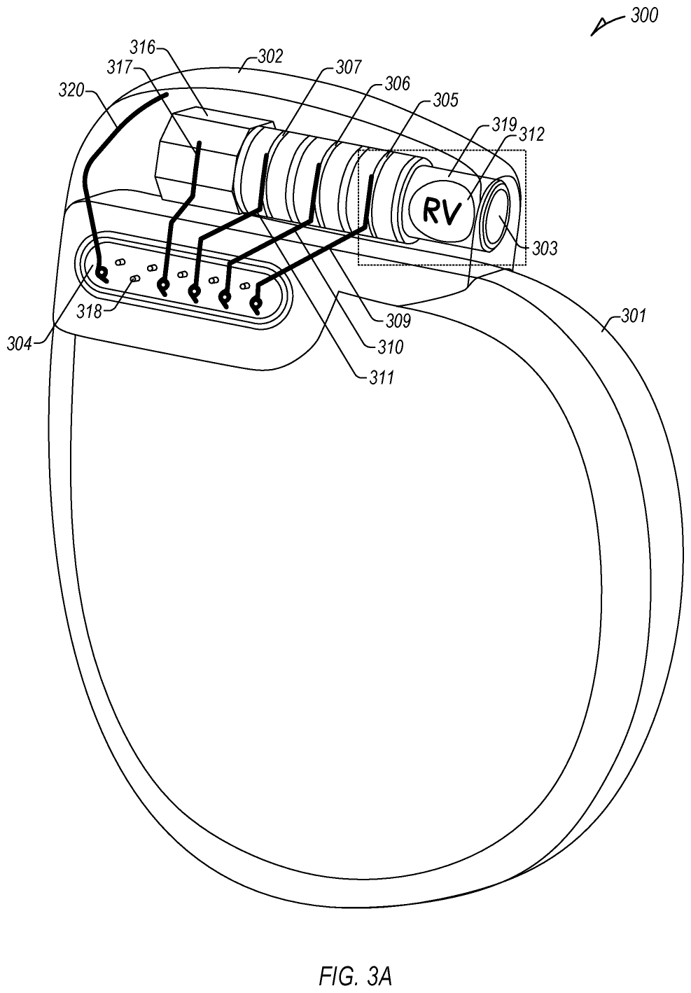

FIG. 3A illustrates an example third implantable medical device 300 including a housing 301 (e.g., commonly referred to as a CAN) and a header 302 coupled to the housing 301, including a first lead port assembly 303 coupled to one or more electronic circuits or electronic circuitry in the housing 301 through a feedthrough 304 on a side of the housing 301. In other examples, the feedthrough 304 can be located on top of or on the other side of the housing 301.

The first lead port assembly 303 (commonly referred to as a bore or a bore assembly) includes a bore opening and electrical connections (e.g., first, second, and third electrical connections 305, 306, 307, etc.) configured to couple to respective electrical connections of a respective lead (not illustrated) once inserted into the bore opening at a proximal end of the first lead port assembly 303 and retained by a retention element (e.g., a set screw or one or more other anchor, retention component, or locking member, etc.) at a distal end of the first lead port assembly 303. In an example, the first, second, and third electrical connections 305, 306, 307 can be parts of separate components (e.g., first, second, and third components corresponding to the first, second, and third electrical connections 305, 306, 307, etc.) that, when coupled or assembled, form a portion of the first lead port assembly 303. The first, second, and third electrical connections 305, 306, 307 can be coupled to one or more posts or connection points (e.g., a connection point 318, etc.) of the feedthrough 304 using respective first, second, and third wires 309, 310, 311 (e.g., pre-formed wires). The third implantable medical device 300 additionally includes an antenna 320 coupled to the feedthrough 304 configured to wirelessly communicate information with one or more other external components.

The first lead port assembly 303 includes a distal component 316 at a distal end of the first lead port assembly 303 having an electrical connection (e.g., in certain examples, the distal component 316 is itself a conductive electrical connection) configured to couple to the feedthrough 304 using a fourth wire 317 (e.g., a pre-formed wire), etc. In certain examples, the distal component 316 can include the retention element, such as on the other (unshown) side of the header 302, etc.

The first lead port assembly 303 includes a strain relief segment 319 at a proximal end of the first lead port assembly 303 configured relieve strain on electrical connections of or associated with the first lead port assembly 303, such as connections of the first, second, and third wires 309, 310, 311 to the feedthrough 304, etc. The strain relief segment 319 can include a tube shaped portion of the first lead port assembly 303 at the proximal end of the first lead port assembly 303 having a first length along the first lead port assembly 303 from a bore opening at the proximal end of the first lead port assembly 303 towards the distal end of the first lead port assembly 303. The bore opening can be commensurate to a diameter of a lead at the strain relief segment 319 when the lead is fully inserted into the first lead port assembly 303.

The first lead port assembly 303 includes a first identifier 312, in this example, a pre-printed label having a specific color (e.g., blue) and text, “RV”, to identify the first lead port 103 as a right ventricle lead port to receive a proximal end of a lead configured to be implanted in or otherwise be associated with a right ventricle of a patient. In other examples, one or more other colors, text, or permutations or combinations of colors and text can be used to identify this or one or more other lead port assemblies.

In certain examples, the first identifier 312 can include an identifier material, separate from (or in the example of laser marking, a component of) the strain relief segment 319 and the first lead port assembly 303, affixed to or printed on the strain relief segment 319 of the first lead port assembly 303 to identify a type of the first lead port assembly 303 (e.g., an RV lead port assembly, an RA lead port assembly, an LV lead port assembly, etc.). In an example, the identifier material can include a carrier material separate from the strain relief segment 319 and the first lead port assembly 303 and affixed to an outer surface of the strain relief segment 319 (e.g., with an adhesive, etc.), the carrier material comprising an identifier (e.g., one or both of color or text, such as a blue color and the text “RV”, etc.) configured to identify the type of the first lead port assembly 303.

FIG. 3B illustrates a larger view of FIG. 3A including the first identifier 312. In an example, the bore opening of the first lead port assembly 303 can have a first diameter, such as 4.7 mm or 4.5 mm or one or more other respective diameters. The strain relief segment 319 has a second larger outer diameter, such as 5.6 mm, etc. The first identifier can have a height 321 and a width 322 of 4 mm, 5 mm, or greater, taking up a substantial portion of the outer diameter of the strain relief segment 319, attaching to a substantial portion of the curve of a side of the outer diameter of the strain relief segment 319. In contrast, the dimensions of the first and second identifiers 112, 115 of FIGS. 1 and 2 are smaller, having a width of 2.5 mm and height of 3 mm. Accordingly, in addition to the processing advantages of the first identifier 312 of FIGS. 3A and 3B, there are readability advantages as well.

Although illustrated on a side portion of the strain relief segment 319, in other examples, the first identifier 312 can be located on a top portion of the strain relief segment 319, opposite a top of the housing of the implantable medical device.

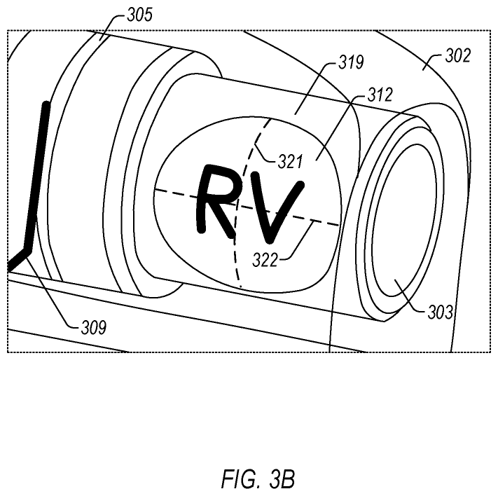

FIG. 4 illustrates example lead configurations 400 in a heart 405, illustrating different lead port configurations for the implantable medical devices illustrated herein. The example lead configurations 400 include first, second, and third leads 420, 425, 430 in traditional lead placements in the right atrium (RA) 406, right ventricle (RV) 407, and in a coronary vein 416 (e.g., the coronary sinus) over the left atrium (LA) 408 and left ventricle (LV) 409, respectively, and a fourth lead 435 positioned in the RV 407 near the His bundle 411, between the AV node 410 and the right and left bundle branches 412, 413 and Purkinje fibers 414, 415. Each lead can be configured to position one or more electrodes or other sensors at various locations in or near the heart 405 to detect physiologic information or provide one or more therapies or stimulation.

The first lead 420, positioned in the RA 406, includes a first tip electrode 421 located at or near the distal end of the first lead 420 and a first ring electrode 422 located near the first tip electrode 421. The second lead 425 (dashed), positioned in the RV 407, includes a second tip electrode 426 located at or near the distal end of the second lead 425 and a second ring electrode 427 located near the second tip electrode 426. The third lead 430, positioned in the coronary vein 416 over the LV 409, includes a third tip electrode 431 located at or near the distal end of the third lead 430 and a third ring electrode 432 located near the third tip electrode 431. The fourth lead 435, positioned in the RV 407 near the His bundle 411, includes a fourth tip electrode 436 located at or near the distal end of the fourth lead 435 and a fourth ring electrode 437 located near the fourth tip electrode 436. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 425 includes a first defibrillation coil electrode 428 located near the distal end of the second lead 425 in the RV 407 and a second defibrillation coil electrode 429 located a distance from the distal end of the second lead 425, such as for placement in or near the superior vena cava (SVC) 417.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 411.

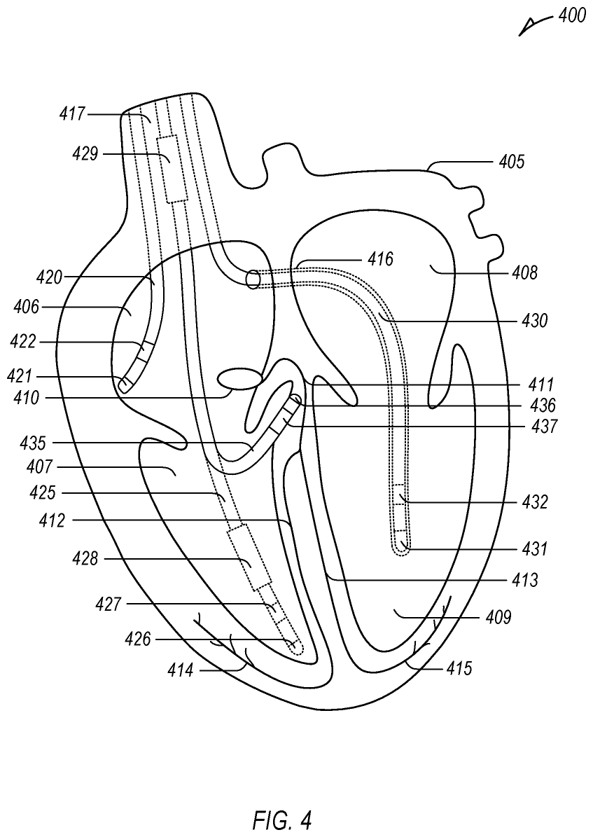

FIG. 5 illustrates an example medical device system 500, such as an implantable medical device, a cardiac rhythm management (CRM) device, etc. In an example, one or more aspects of the example system 500 can be a component of, or communicatively coupled to, an implantable medical device, an insertable cardiac monitor, etc. The system 500 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 500 can include a single medical device or a plurality of medical devices implanted in a body of a patient or otherwise positioned on or about the patient to monitor patient physiologic information of the patient using one or more sensors, such as a sensor 501. In an example, the sensor 501 can include one or more of: a respiration sensor configured to receive respiration information (e.g., a respiration rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., intrathoracic impedance sensor, transthoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 500 can include a signal receiver circuit 502 and an assessment circuit 503. The signal receiver circuit 502 can be configured to receive physiologic information of a patient (or group of patients) from the sensor 501. The assessment circuit 503 can be configured to receive information from the signal receiver circuit 502, and to determine one or more parameters (e.g., physiologic parameters, stratifiers, etc.) or existing or changed patient conditions using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 503 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

The assessment circuit 503 can be configured to provide an output to a user, such as to a display or one or more other user interface, the output including a score, a trend, an alert, or other indication. In other examples, the assessment circuit 503 can be configured to provide an output to another circuit, machine, or process, such as a therapy circuit 504 (e.g., a cardiac resynchronization therapy (CRT) circuit, a chemical therapy circuit, etc.), etc., to control, adjust, or cease a therapy of a medical device, a drug delivery system, etc., or otherwise alter one or more processes or functions of one or more other aspects of a medical-device system, such as one or more cardiac resynchronization therapy parameters, drug delivery, dosage determinations or recommendations, etc. In an example, the therapy circuit 504 can include one or more of a stimulation control circuit, a cardiac stimulation circuit, a neural stimulation circuit, a dosage determination or control circuit, etc. In other examples, the therapy circuit 504 can be controlled by the assessment circuit 503, or one or more other circuits, etc.

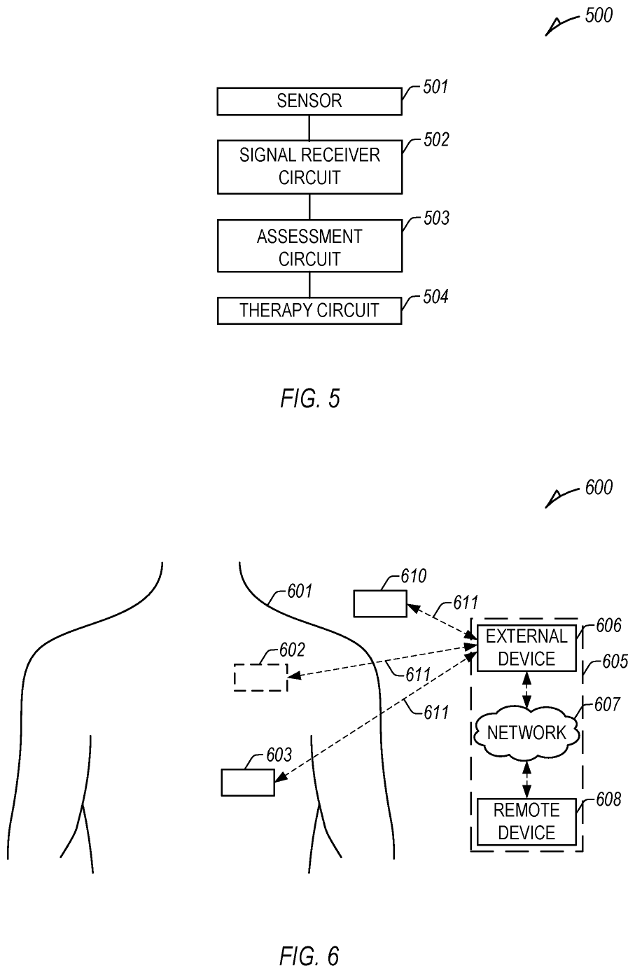

FIG. 6 illustrates an example patient management system 600 and portions of an environment in which the patient management system 600 may operate. The patient management system 600 can perform a range of activities, including remote patient monitoring and diagnosis of a disease condition. Such activities can be performed proximal to a patient 601, such as in a patient home or office, through a centralized server, such as in a hospital, clinic, or physician office, or through a remote workstation, such as a secure wireless mobile computing device.

The patient management system 600 can include one or more ambulatory medical devices, an external system 605, and a communication link 611 providing for communication between the one or more ambulatory medical devices and the external system 605. The one or more ambulatory medical devices can include an implantable medical device (IMD) 602, a wearable medical device 603, or one or more other implantable, leadless, subcutaneous, external, wearable, or ambulatory medical devices configured to monitor, sense, or detect information from, determine physiologic information about, or provide one or more therapies to treat various conditions of the patient 601, such as one or more cardiac or non-cardiac conditions (e.g., dehydration, sleep disordered breathing, etc.).

In an example, the implantable medical device 602 can include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 601. In another example, the implantable medical device 602 can include a monitor implanted, for example, subcutaneously in the chest of patient 601, the implantable medical device 602 including a housing containing circuitry and, in certain examples, one or more sensors, such as a temperature sensor, etc.

Traditional cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 602 can include an assessment circuit configured to detect or determine specific physiologic information of the patient 601, or to determine one or more conditions or provide information or an alert to a user, such as the patient 601 (e.g., a patient), a clinician, or one or more other caregivers or processes, such as described herein. The implantable medical device 602 can alternatively or additionally be configured as a therapeutic device configured to treat one or more medical conditions of the patient 601. The therapy can be delivered to the patient 601 via the lead system and associated electrodes or using one or more other delivery mechanisms. The therapy can include delivery of one or more drugs to the patient 601, such as using the implantable medical device 602 or one or more of the other ambulatory medical devices, etc. In some examples, therapy can include cardiac resynchronization therapy for rectifying dyssynchrony and improving cardiac function in heart failure patients. In other examples, the implantable medical device 602 can include a drug delivery system, such as a drug infusion pump to deliver drugs to the patient for managing arrhythmias or complications from arrhythmias, hypertension, hypotension, or one or more other physiologic conditions. In other examples, the implantable medical device 602 can include one or more electrodes configured to stimulate the nervous system of the patient or to provide stimulation to the muscles of the patient airway, etc.

The wearable medical device 603 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 605 can include a dedicated hardware/software system, such as a programmer, a remote server-based patient management system, or alternatively a system defined predominantly by software running on a standard personal computer. The external system 605 can manage the patient 601 through the implantable medical device 602 or one or more other ambulatory medical devices connected to the external system 605 via a communication link 611. In other examples, the implantable medical device 602 can be connected to the wearable medical device 603, or the wearable medical device 603 can be connected to the external system 605, via the communication link 611. This can include, for example, programming the implantable medical device 602 to perform one or more of acquiring physiologic data, performing at least one self-diagnostic test (such as for a device operational status), analyzing the physiologic data, or optionally delivering or adjusting a therapy for the patient 601. Additionally, the external system 605 can send information to, or receive information from, the implantable medical device 602 or the wearable medical device 603 via the communication link 611. Examples of the information can include real-time or stored physiologic data from the patient 601, diagnostic data, such as detection of patient hydration status, hospitalizations, responses to therapies delivered to the patient 601, or device operational status of the implantable medical device 602 or the wearable medical device 603 (e.g., battery status, lead impedance, etc.). The communication link 611 can be an inductive telemetry link, a capacitive telemetry link, or a radio-frequency (RF) telemetry link, or wireless telemetry based on, for example, “strong” Bluetooth or IEEE 602.11 wireless fidelity “Wi-Fi” interfacing standards. Other configurations and combinations of patient data source interfacing are possible.

The external system 605 can include an external device 606 in proximity of the one or more ambulatory medical devices, and a remote device 608 in a location relatively distant from the one or more ambulatory medical devices, in communication with the external device 606 via a communication network 607. Examples of the external device 606 can include a medical device programmer. The remote device 608 can be configured to evaluate collected patient or patient information and provide alert notifications, among other possible functions. In an example, the remote device 608 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 608 can receive data from multiple patients. The data can be collected by the one or more ambulatory medical devices, among other data acquisition sensors or devices associated with the patient 601. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 608 may additionally include one or more locally configured clients or remote clients securely connected over the communication network 607 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 608, including the server and the interconnected clients, may also execute a follow-up scheme by sending follow-up requests to the one or more ambulatory medical devices, or by sending a message or other communication to the patient 601 (e.g., the patient), clinician or authorized third party as a compliance notification.

The communication network 607 can provide wired or wireless interconnectivity. In an example, the communication network 607 can be based on the Transmission Control Protocol/Internet Protocol (TCP/IP) network communication specification, although other types or combinations of networking implementations are possible. Similarly, other network topologies and arrangements are possible.

One or more of the external device 606 or the remote device 608 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 606 or the remote device 608 can include a respective display unit for displaying the physiologic or functional signals, or alerts, alarms, emergency calls, or other forms of warnings to signal the detection of arrhythmias. In some examples, the external system 605 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 605 can be implemented using hardware, software, firmware, or combinations thereof. Portions of the one or more ambulatory medical devices or the external system 605 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The patient management system 600 can include a therapy device 610, such as ambulatory or external therapy device configured to send information to or receive information from one or more of the ambulatory medical devices or the external system 605 using the communication link 611. In an example, the one or more ambulatory medical devices, the external device 606, or the remote device 608 can be configured to control one or more parameters of the therapy device 610. The external system 605 can allow for programming the one or more ambulatory medical devices and can receives information about one or more signals acquired by the one or more ambulatory medical devices, such as can be received via a communication link 611. The external system 605 can include a local external implantable medical device programmer. The external system 605 can include a remote patient management system that can monitor patient status or adjust one or more therapies such as from a remote location.

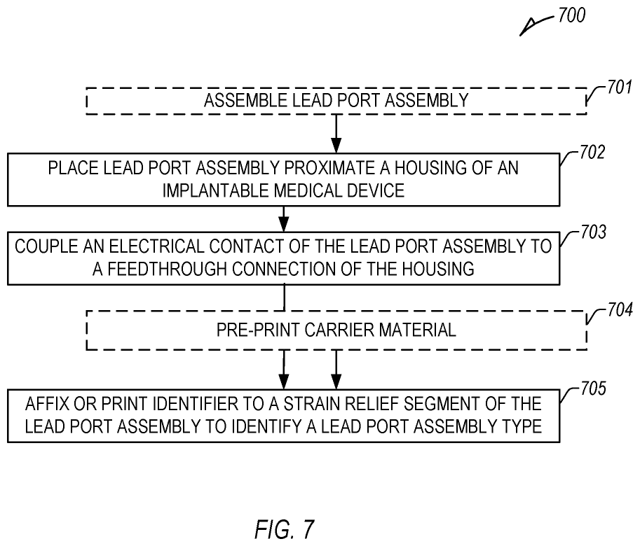

FIG. 7 illustrates an example method 700 of identifying a type of lead port assembly in a header of an implantable medical device. At step 701, a lead port assembly is assembled. The lead port assembly can be a generic lead port assembly, manufactured and stocked without specific labeling until ready for use or inclusion as a specific lead port assembly in a header of an implantable medical device. In certain examples, the type of lead port assembly depends at least in part on electrical connections between the lead port assembly and the implantable medical device.

At step 702, the lead port assembly can be placed proximate to a housing of an implantable medical device, such as during assembly or manufacturing of the implantable medical device. At step 703, one or more electrical contacts of the lead port assembly can be coupled to a feedthrough connection on the housing of the implantable medical device, etc. In an example, placement can include as part of an inline manufacturing or assembly process, where an otherwise generic lead port assembly having a number of electrical configurations, size, etc., can be placed for inclusion in an implantable medical device.

At step 704, an identifier can be printed on a carrier material, such as a polyester or other carrier or backing material (e.g., paper, plastic film, cloth, foil, foam, etc.). In certain examples, the carrier material can include an adhesive opposite the printed identifier, such as to retain the carrier material on a desired surface.

At step 705, the identifier can be affixed to or printed on a strain relief or other segment of the lead port assembly, such as to identify a type of the lead port assembly after inclusion in the header of the implantable medical device. In an example, the printed carrier material at step 704 can be placed on the lead port assembly, such as using a pick and place machine or process, as part of an inline manufacturing process of the implantable medical device, before or after placement of one or more wires between the electrical contacts of the lead port assembly to the housing or the feedthrough, etc. In other examples, the identifier can be laser printed on the lead bore assembly, or in certain examples, ink jet printed using one or more additional masking procedures, etc.

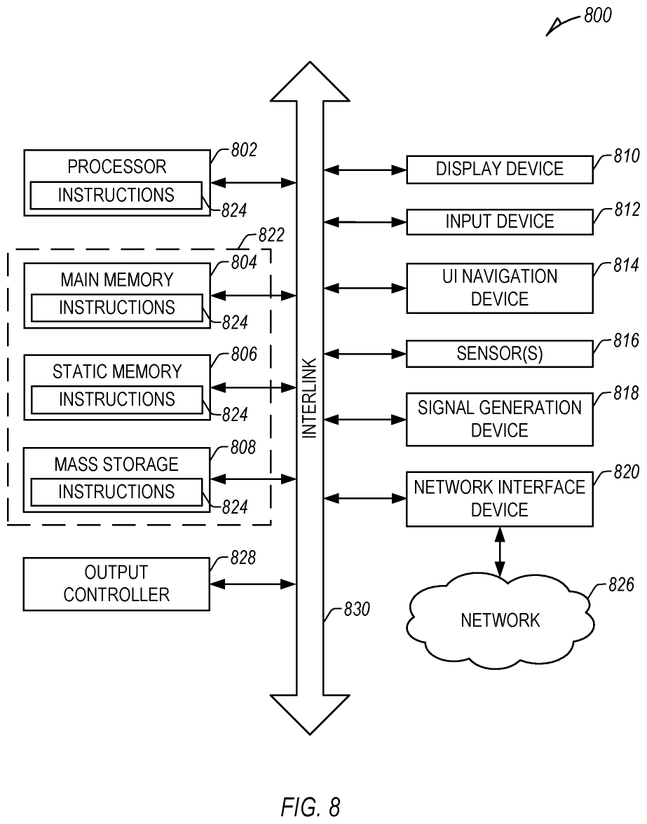

FIG. 8 illustrates a block diagram of an example machine 800 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 800. Circuitry (e.g., processing circuitry, an assessment circuit, etc.) is a collection of circuits implemented in tangible entities of the machine 800 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 800 follow.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 800 may include a hardware processor 802 (e.g., a central processing unit (CPU), a graphics processing unit (GPU), a hardware processor core, or any combination thereof), a main memory 804, a static memory (e.g., memory or storage for firmware, microcode, a basic-input-output (BIOS), unified extensible firmware interface (UEFI), etc.) 806, and mass storage 808 (e.g., hard drive, tape drive, flash storage, or other block devices) some or all of which may communicate with each other via an interlink (e.g., bus) 830. The machine 800 may further include a display unit 810, an input device 812 (e.g., a keyboard), and a user interface (UI) navigation device 814 (e.g., a mouse). In an example, the display unit 810, input device 812, and UI navigation device 814 may be a touch screen display. The machine 800 may additionally include a signal generation device 818 (e.g., a speaker), a network interface device 820, and one or more sensors 816, such as a global positioning system (GPS) sensor, compass, accelerometer, or one or more other sensors. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 802, the main memory 804, the static memory 806, or the mass storage 808 may be, or include, a machine-readable medium 822 on which is stored one or more sets of data structures or instructions 824 (e.g., software) embodying or utilized by any one or more of the techniques or functions described herein. The instructions 824 may also reside, completely or at least partially, within any of registers of the hardware processor 802, the main memory 804, the static memory 806, or the mass storage 808 during execution thereof by the machine 800. In an example, one or any combination of the hardware processor 802, the main memory 804, the static memory 806, or the mass storage 808 may constitute the machine-readable medium 822. While the machine-readable medium 822 is illustrated as a single medium, the term “machine-readable medium” may include a single medium or multiple media (e.g., a centralized or distributed database, and/or associated caches and servers) configured to store the one or more instructions 824.

The term “machine-readable medium” may include any medium that is capable of storing, encoding, or carrying instructions for execution by the machine 800 and that cause the machine 800 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may be further transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 800, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system, comprising:
a lead port assembly for placement in a header of an implantable medical device, the lead port assembly comprising:
proximal and distal ends, the proximal end comprising a bore configured to receive a proximal end of a lead; and
an electrical contact configured to couple to an electrical contact of the lead when the lead is inserted into the lead port assembly; and
an identifier material, separate from the lead port assembly, affixed to or printed on the lead port assembly, comprising a marking to identify a type of the lead port assembly.

2. The system of claim 1, wherein the lead port assembly comprises a strain relief segment at the proximal end of the bore having a first length,
wherein the electrical contact is located distal to the strain relief segment on the lead port assembly,
wherein the identifier material is separate from the strain relief segment and the lead port assembly, and affixed to or printed on the strain relief segment of the lead port assembly to identify the type of the lead port assembly.

3. The system of claim 2, wherein the identifier material includes a carrier material comprising the marking,
wherein the carrier material is separate from the strain relief segment and the lead port assembly and is affixed to the strain relief segment to identify the type of the lead port assembly.

4. The system of claim 3, wherein the carrier material is affixed to an outer surface of the strain relief segment with an adhesive.

5. The system of claim 2, wherein the identifier material is affixed to or printed on a side portion of the strain relief segment viewable, through material of the header, from a side of the header.

6. The system of claim 2, wherein the identifier material is affixed to or printed on a top portion of the strain relief segment,
wherein the top portion of the strain relief segment is opposite a top of a housing of the implantable medical device, between a top of the header and the top of the housing.

7. The system of claim 2, wherein the strain relief segment comprises a tube shape at the proximal end of the bore having an opening commensurate to a diameter of the lead at the strain relief segment when the lead is fully inserted into the lead port assembly to relieve strain on electrical connections of or associated with the lead port assembly,
wherein the first length is along the lead port assembly from the proximal end of the bore.

8. The system of claim 1, wherein the lead port assembly comprises a distal end to contact the proximal end of the lead when the lead is fully inserted into the lead port assembly,
wherein the distal end comprises a mechanical retention feature to engage and retain the proximal end of the lead when the lead is fully inserted into the lead port assembly.

9. The system of claim 1, comprising the header and a housing of the implantable medical device,
wherein the lead port assembly is a generic lead port assembly for placement in the header,
wherein the identifier material is affixed to or printed on the lead port assembly to identify the type of the lead port assembly after the lead port assembly is coupled to a housing of the implantable medical device before incorporation in the header,
wherein the type of the lead port assembly depends at least in part on electrical connections between the lead port assembly and the implantable medical device.

10. A method, comprising:
affixing an identifier material to or printing the identifier material on a lead port assembly for placement in a header of an implantable medical device,
wherein the lead port assembly comprises:
proximal and distal ends, the proximal end comprising a bore configured to receive a proximal end of a lead; and
an electrical contact configured to couple to an electrical contact of the lead when the lead is inserted into the lead port assembly,
wherein the identifier material comprises a marking to identify a type of the lead port assembly.

11. The method of claim 10, wherein the lead port assembly comprises a strain relief segment at the proximal end of the bore having a first length,
wherein the electrical contact is located distal to the strain relief segment on the lead port assembly,
wherein the identifier material is separate from the strain relief segment and the lead port assembly,
wherein affixing the identifier material to or printing the identifier material on the lead port assembly comprises affixing the identifier material to or printing the identifier material on the strain relief segment of the lead port assembly to identify the type of the lead port assembly.

12. The method of claim 11, wherein affixing the identifier material to or printing the identifier material on the strain relief segment of the lead port assembly comprises affixing the identifier material to or printing the identifier material on the strain relief segment of the lead port assembly prior to forming the header of the implantable medical device.

13. The method of claim 12, wherein the identifier material includes a carrier material comprising the marking,
wherein the carrier material is separate from the strain relief segment and the lead port assembly,
wherein affixing the identifier material to or printing the identifier material on the strain relief segment of the lead port assembly comprises printing the marking on the carrier material and affixing the carrier material to the strain relief segment of the lead port assembly to identify the type of the lead port assembly.

14. The method of claim 13, wherein affixing the carrier material to the strain relief segment comprises affixing the carrier material to an outer surface of the strain relief segment using an adhesive.

15. The method of claim 12, wherein affixing the identifier material to or printing the identifier material on the strain relief segment of the lead port assembly comprises affixing the identifier material to or printing the identifier material on a side portion of the strain relief segment viewable through material of the header from a side of a header.

16. The method of claim 11, wherein affixing the identifier material to or printing the identifier material on the strain relief segment of the lead port assembly comprises affixing the identifier material to or printing the identifier material on a top portion of the strain relief segment,
wherein the top portion of the strain relief segment is opposite a top of a housing of the implantable medical device, between a top of the header and the top of the housing.

17. The method of claim 11, wherein the strain relief segment comprises a tube shape at the proximal end of the bore having an opening commensurate to a diameter of the lead at the strain relief segment when the lead is fully inserted into the lead port assembly to relieve strain on electrical connections of or associated with the lead port assembly,
wherein the first length is along the lead port assembly from the proximal end of the bore.

18. The method of claim 10, wherein the lead port assembly comprises a distal end to contact the proximal end of the lead when the lead is fully inserted into the lead port assembly,
wherein the distal end comprises a mechanical retention feature to engage and retain the proximal end of the lead when the lead is fully inserted into the lead port assembly.

19. The method of claim 10, wherein the lead port assembly is a generic lead port assembly for placement in the header,
wherein affixing the identifier material to or printing the identifier material on the lead port assembly comprises affixing the identifier material to or printing the identifier material on the lead port assembly after the lead port assembly is coupled to a housing of the implantable medical device before incorporation in the header.

20. A method, comprising:
identifying a type of a lead port assembly for placement in a header of an implantable medical device using a marking on an identifier material affixed to or printed on the lead port assembly,
wherein the lead port assembly comprises:
proximal and distal ends, the proximal end comprising a bore configured to receive a proximal end of a lead; and
an electrical contact configured to couple to an electrical contact of the lead when the lead is inserted into the lead port assembly.
